(19) United States
(12) Patent Application Publication
(10) Pub. No.: US 2022/0157452 A1
(43) Pub. Date: May 19, 2022

(54) Method for Providing Data for an Interface

(71) Applicant: Cosinuss GmbH, Munich (DE)

(72) Inventor: Johannes Kreuzer, München (DE)

(21) Appl. No.: 17/309,967

(22) PCT Filed: Jan. 7, 2020
(86) PCT No.: PCT/EP2020/050227

§ 371 Date: Jan. 26, 2022

(30) Foreign Application Priority Data
Jan. 7, 2019 (DE) 10 2019 200 100.9

(51) Int. Cl. G16H 40/67; H04R 25/00; G16H 50/30; A61B 5/0205; A61B 5/021; A61B 5/024; A61B 5/0531; A61B 5/08; A61B 5/1455; A61B 5/145; A61B 5/333; A61B 5/384; A61B 5/00

(52) U.S. Cl. CPC G16H 40/67; A61B 2562/0271; H04R 25/554; G16H 50/30; A61B 5/02055; A61B 5/021; A61B 5/02416; A61B 5/0531; A61B 5/0803; A61B 5/0816; A61B 5/14551; A61B 5/14532; A61B 5/333; A61B 5/384; A61B 5/6817; A61B 5/7475; A61B 5/0006; A61B 5/0008; H04R 2225/55; A61B 2562/0247; H04R 25/609

(57) ABSTRACT

The invention relates to a method for providing data for an interface, wherein the interface is configured for computer-implemented health monitoring by means of at least one electronic apparatus integrated in a hearing aid, to a data processing system comprising means for carrying out the method, and to a computer program and a computer-readable storage medium comprising instructions for carrying out the method.

### TECHNICAL FIELD

The invention relates to a method for providing data for an interface, wherein the interface is configured for computer-implemented health monitoring by means of at least one electronic apparatus integrated in a hearing aid, according to claim 1. The invention further relates to a data processing system according to claim 16, to a computer program according to claim 17, and to a computer-readable storage medium according to claim 18.

### PRIOR ART

Methods for providing data for an interface are known from the prior art. In particular, such methods and the corresponding interfaces are known in systems for computer-implemented health monitoring. Also known from the prior art are hearing aids, by means of which individual health parameters or health aspects of a wearer of a hearing aid can be monitored via at least one electronic apparatus integrated in the hearing aid.

Disadvantageously, some of the monitoring systems are implemented in a technically complicated manner and comprise a large number of individual components. EP 3035710 A1 describes a system for monitoring the status and/or performance of one or more hearing aids, wherein the system comprises multiple access points for receiving wirelessly transmitted signals, which in turn are communicatively connected to a central processing unit; finally, the central processing unit is connected to the internet/cloud. US 2013/0343585 A1 describes a method for a hearing aid device comprising a medical sensor, wherein functions in the hearing aid device can be executed in multiple communication steps on the basis of recorded sensor data.

### Presentation of the Invention, Problem, Solution, Advantages

Proceeding from the aforementioned methods and apparatuses of the prior art, the problem addressed by the present invention is that of providing a method and systems and programs associated with the method that do not have said disadvantages. In particular, a method is intended to make it possible to provide data using only a few method steps, and the associated systems are intended to be technically easy to implement.

The method according to the invention is intended to enable easy interaction of a user of the method according to the invention with an interface, so that different configurations of a sensor arrangement, arranged in a hearing aid, can be selected in one step and the health-related data recorded in a time interval can easily be transmitted to the interface.

The problem addressed by the invention is solved by the method and the associated systems and programs defined in the claims, as will also become clear from the included exemplary embodiment.

In a first aspect, therefore, the invention relates to a method for providing data for an interface, wherein the interface is configured for computer-implemented health monitoring by means of at least one electronic apparatus integrated in a hearing aid, which method comprises multiple steps, wherein in a first step an arrangement of one or more sensors S₁-Sᵢ, which are arranged in a hearing aid and are designed to record health-related data, is provided for selection by a user of the health-related data. Hearing aids for treating the most common form of hearing loss, sensorineural hearing loss, are known from the prior art. These hearing aids are designed as Behind-The-Ear and In-The-Ear devices, the latter being available in a variety of different forms with varying degrees of structural miniaturization. According to the invention, the term “health-related data” will be understood to mean physiological data of a wearer of the hearing aid that can be recorded by means of external sensors, for example data associated with heart rate or blood pressure. According to the invention, one or more sensors S₁-Sᵢ are arranged in the hearing aid. However, the method according to the invention may also take into account data recorded by additional sensors, the additional sensors recording data at other, external points of measurement on the wearer of the hearing aid, for example on the wrist via a suitable wrist band or on the chest via a suitable chest strap. The method according to the invention may also take into account data recorded by additional, internally located sensors. In a second step of the method according to the invention, a user's choice of arrangement is recorded, it being possible for the user and the wearer of the hearing aid to be different people. By way of example, from a sensor arrangement of the sensors S₁-Sᵢ, the user's choice of sensors S₁, S₂ and S₄ is recorded. The selected sensors may record in different ways physiological data associated with a health parameter, for example the health parameter “heart rate” via different modalities of the sensors in question. The selected sensors may also record physiological data of different modalities, for example heart rate and body temperature. In a further step, the health-related data of a wearer of the hearing aid are recorded by means of the one or more sensors, selected by the user, in a time interval. The time interval may be preset, for example as a function of the type of health-related data (for example recording multiple ECG intervals), or may be suitably selected by a user of the method according to the invention. In the last method step, the recorded data are transmitted to an interface. An interface will preferably be understood here to mean a software interface, the software interface being a logical point of contact in a software system, via which the exchange of commands and data between different processes and components is enabled and controlled. Such interfaces are known from the prior art and are in principle differentiated into data-oriented interfaces, which are only used for communication, and function-oriented interfaces for executing certain functionalities, which primarily synchronize or support involved system parts.

The method according to the invention enables easy configuration, by a user, of the sensors arranged in a hearing aid of a wearer for recording health-related data. By way of example, health-related data of a wearer can be recorded at a first location and can be transmitted to a user at a second location for analysis purposes. The method according to the invention also enables the simultaneous configuration and recording of health-related data by multiple users, optionally at different locations.

In one preferred development of the method according to the invention, the sensor data may be recorded at a sampling rate of up to 1 kHz. The transmission may take place at a transmission rate of up to 1 kHz. The sampling rate of up to 1 kHz enables a recording of health-related data that permits more meaningful information to be deduced with regard to physiological parameters derived therefrom, for example the blood pressure from the pressure curve recorded by means of a pressure sensor. The transmission of the data to the interface may take place in a continuous manner or in packets.

In a further implementation of the method according to the invention, the health-related data may be selected from the group comprising electrocardiogram and/or heart rate and/or pulse rate and/or blood pressure and/or body temperature and/or skin conductivity and/or blood sugar level and/or electrical brain activity and/or oxygen saturation and/or respiratory rate. The respective health-related, physiological data are known from the prior art. By means of the method according to the invention, different health-related data of the wearer of the hearing aid can easily be simultaneously or successively transmitted to an interface and can then be retrieved by one or more users of the method according to the invention.

In one preferred embodiment, the sensors S₁-Sᵢ may be selected from the group comprising PPG sensor, ECG/EEG sensor, pressure sensor, temperature sensor, conductivity sensor, moisture sensor, glucose sensor. A conventional PPG sensor (photoplethysmography, PPG) lies directly on the skin and consists of a light source, which emits light of a certain application wavelength that passes through tissue and blood vessels, and a photodetector. A conventional ECG (electrocardiogram) sensor may be designed with at least two small electrodes for recording electrical heart signals; a conventional EEG (electroencephalography) sensor may be designed with at least two small electrodes for recording brain waves; a temperature sensor records the body temperature through the infrared radiation of the tissue or through a contact temperature sensor. All the aforementioned sensors are known from the prior art and are or can be miniaturized in such a way that they can be arranged in a conventional hearing aid of the type mentioned above and can record health-related data of a wearer of the hearing aid with as little interference as possible. In particular, the sensors S₁-Sᵢ may preferably be arranged in a hearing aid to be worn behind the ear, as a result of which the interaction between the separate sensor signals is minimized.

In one advantageous development of the method according to the invention, in a further step the recorded data may be recorded in a memory integrated in the hearing aid. The storage medium is known from the prior art and may comprise one or more types of memory, including RAM or Flash, etc., it being possible for the memory to be volatile or non-volatile. By means of such a memory, data recorded in one or more past time intervals can also be transmitted to the interface and retrieved by the user.

In a further embodiment, the choice of arrangement may be made via the interface, and the choice of arrangement may reach the interface via wireless communication from an external computer device connected to the hearing aid, The wireless communication may in this case be achieved by means of conventional communication technology, for example via Bluetooth or ANT+ or WiFi or NFC, or via radio access technology (RAT), such as for example NOMA (non-orthogonal multiple access), or via a suitably constructed, RAT-based communication architecture comprising different stations, which combines low signal interference with seamless transmission and high capacity.

In one preferred development, the method may be able to be automated according to the user's specifications. The method can thus be executed at specified points in time, so that data from the user are transmitted calculably for the interface, for example once per hour, or once per day.

In a further implementation of the method according to the invention, the method may be executed automatically when an activity state of the wearer changes, wherein the activity state of the wearer is continuously recorded via an acceleration sensor. Acceleration sensors designed as a micro-electromechanical system (MEMS) are known from the prior art in the field of medical monitoring. MEMS are characterized by a high degree of reliability and, due to their small size, by a high measurement speed.

In a further implementation of the method according to the invention, following the step of recording health-related data of a wearer of the hearing aid by means of the one or more sensors, selected by the user, in a time interval, the method may comprise additional steps, namely first determining, from the sensor data of two or more sensors S₁, S₂-Sᵢ, a data pattern D₁, D₂-Dᵢ of the wearer of the hearing aid, wherein each sensor S₁, S₂-Sᵢ is associated with a corresponding sensor index SI₁, SI₂-SIᵢ. By means of calculation means, at least one health indicator of the wearer of the hearing aid can be calculated, namely as a function of the at least one data pattern and a multidimensional pattern of health-related data, wherein a first dimension is time and at least a second dimension is the sensor index SI₁, SI₂-SIᵢ, wherein the health indicator and/or the data pattern and/or the recorded data are transmitted to the interface. According to the invention, a health indicator will be understood to mean an indication of a certain state of a health parameter of the wearer of the hearing aid. By way of example, a health indicator relating to the blood pressure of the wearer may indicate that the blood pressure is in the normal range, or else that it is too high (hypertension) or too low (hypotension) with regard to the measured time interval. In a further step, it can be checked, by means of checking means, whether the calculated health indicator is plausible, namely when sensor data from sensors S₁, S₂-Sᵢ with different modalities are used. By way of example, a heart rate of the wearer of the hearing aid can be determined by means of the data from a PPG sensor or from an ECG sensor or from a pressure sensor. If different, implausible values for the health indicator heart rate are determined from the data in question, the lack of plausibility can be signaled to the wearer of the hearing aid and/or to the user of the method via suitable communication means. In particular, the at least one health indicator may preferably indicate a measured value of the quality of the measured values and of the health of the wearer in the time interval.

In one particularly preferred implementation of the method according to the invention, the multidimensional pattern of health-related data may be a wearer-specific pattern, which can be generated by a sequence of method steps. First, the sensor data of a wearer of a hearing aid are recorded in a first time interval, wherein the sensor data are data from one or more sensors S₁-Si, arranged in a hearing aid of a wearer, for recording health-related data. In a subsequent step, a respective data pattern D₁-Dᵢ of the wearer in the first time interval can be determined from these sensor data of the one or more sensors S₁-Sᵢ, followed by the step of recording sensor data of the wearer of a hearing aid in at least one further time interval. A further, respective data pattern D₁-Dᵢ of the wearer in the at least one further time interval can be determined from the sensor data of the one or more sensors S₁-Sᵢ. By means of calculation means, an average data pattern DD₁-DDᵢ of the user can then be calculated from the data patterns determined in the preceding determination steps in order to obtain the wearer-specific, multidimensional pattern of health-related data of the wearer. The aforementioned method for generating the multidimensional data pattern enables optimal adaptation of the health indicator to the wearer. This is advantageous, for example, when the wearer-specific multidimensional data pattern deviates from a multidimensional data pattern derived from generally available health data in a way that, although significant, is not pathological for the wearer and thus has no disease relevance. In an alternative embodiment, the multidimensional pattern of health-related data may be a reference table of general, physiological data. Such a table relates to health-related data from a large number of people and contains normal or average values, which are sorted for example with regard to age and gender.

In one particularly preferred implementation of the method according to the invention, calculation means may be arranged in the hearing aid, in a mobile computer unit and/or in a central processing unit; with particular preference, calculation means may be arranged in the hearing aid or in a mobile computer unit; most preferably, calculation means may be arranged in a mobile computer unit. If calculation means are arranged in the hearing aid, some of the data processing may advantageously already take place in the hearing aid; in particular, however, the arrangement of the calculation means depends on the number and dimensioning of the sensors since, in addition to the sensors, the communication structures and the data processing structures, devices that supply power must accordingly also be provided. Preferably, the hearing aid power supply can be used for the additional sensors and for transmission; alternatively, the power is provided by a local power-generating device for supplying power to sensors and structures, for example in the form of a miniaturized, thermoelectric generator. In one advantageous development of the method according to the invention, the interface may be arranged in a mobile computer unit and/or in a central processing unit.

In a further embodiment of the method according to the invention, the method may comprise an authentication step. Authentication will be understood here to mean a verification of the claim of authenticity, by means of which the user of the method indicates whether he is authorized to access the data. By way of example, the user may first specify a username and additionally authenticate himself by specifying a password. Advantageously, the authentication may take place at an early point in the method, namely following the recording and prior to the transmission of the health-related data to the interface. Alternatively, the authentication may also take place following the transmission of the recorded data. Further protection of the health-related data of the wearer of the hearing aid can be achieved by encrypting the data; in this case, the data may advantageously be encrypted prior to transmission of the data to the interface.

In a second aspect, the invention relates to a data processing system comprising means for executing the above-described method according to the invention. A data processing system will be understood here to mean a computer, a computing machine or a computing system. By definition, a data processing system is a functional unit for processing data, processing being defined as the performance of mathematical, transforming, transmitting or storing operations. The data processing system according to the present invention may for example comprise various selection means, recording means, transmission means and/or calculation means.

In a third aspect, the invention relates to a computer program comprising instructions which, when the program is run on a computer, cause the computer to execute the method described above.

In a fourth aspect, the invention relates to a computer-readable storage medium comprising instructions which, when the program is run on a computer, cause the computer to execute the method described above.

A preferred exemplary embodiment is described, but the invention is not limited thereto. In principle, any variant of the invention described or implied in the context of the present application may be particularly advantageous, depending on the economic, technical and optionally medical requirements in each case. Unless otherwise stated, and insofar as is technically feasible in principle, individual features of the described embodiments are interchangeable or can be combined with one another and also with features known per se from the prior art.

### BRIEF DESCRIPTION OF THE FIGURE

FIG. 1 shows a flowchart of the method according to the invention.

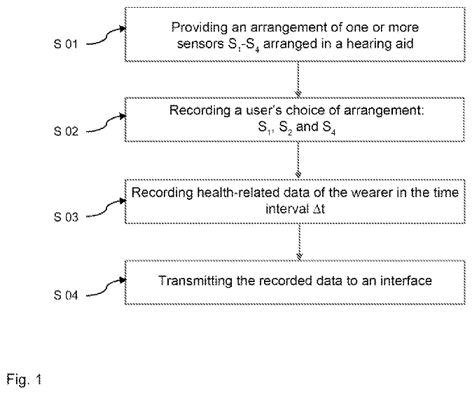

### DETAILED DESCRIPTION

The invention relates to a method for providing data for an interface, wherein the interface is configured for computer-implemented health monitoring by means of at least one electronic apparatus integrated in a hearing aid. The method comprises multiple steps, wherein in a first step (S 01) an arrangement of one or more sensors, in this case for example sensors S₁-S₄, which are arranged in a hearing aid and are designed to record health-related data, is provided for selection by a user of the health-related data. S₁-S₄ may be designed for example as a PPG sensor, a pressure sensor, a temperature sensor and a conductivity sensor. Accordingly, in the present case, health-related data can be recorded in relation to oxygen saturation, blood pressure, body temperature and skin conductivity. All the aforementioned sensors are already known in miniaturized form from the prior art, so that they can be arranged in a conventional hearing aid, for example in a hearing aid to be worn behind the ear for treating sensorineural hearing loss, and can record health-related data of a wearer of the hearing aid with as little interference as possible. In a second step (S 02), a user's choice of arrangement is recorded; by way of example, from a sensor arrangement of the sensors S₁-S₄, the sensors S₁, S₂ and S₄ are selected. In a third step (S 03), the health-related data of a wearer of the hearing aid are recorded by means of the one or more sensors, selected by the user, in a time interval Δt. The time interval may be preset, for example as a function of the type of health-related data, or may be suitably selected by a user of the method according to the invention. In the fourth step (S 04), the recorded data are transmitted to an interface. An interface will preferably be understood here to mean a software interface, the software interface being a logical point of contact in a software system that enables and controls the exchange of commands and data between different processes and components.

1. A method for providing data for an interface, wherein the interface is configured for computer-implemented health monitoring by means of at least one electronic apparatus integrated in a hearing aid, which method comprises:
01. providing an arrangement of one or more sensors S₁-Sᵢ, which are arranged in a hearing aid and are designed to record health-related data, for selection by a user of the health-related data,
02. recording a user's choice of arrangement,
03. recording health-related data of a wearer of the hearing aid as recorded data by means of the one or more sensors, selected by the user, in a time interval,
04. transmitting the recorded data to an interface.

2-18. (canceled)

19. The method according to claim 1, wherein health-related data from the one or more sensors are recorded at a sampling rate of up to 1 kHz, and wherein transmitting the recorded data takes place at a transmission rate of up to 1 kHz.

20. The method according to claim 1, wherein the health-related data are comprised of one or more of the group comprising electrocardiogram, heart rate, pulse rate, blood pressure, body temperature, skin conductivity, blood sugar level, electrical brain activity, oxygen saturation, and respiratory rate.

21. The method according to claim 1, wherein the sensors S₁-Sᵢ are selected from the group comprising PPG sensor, ECG/EEG sensor, pressure sensor, temperature sensor, conductivity sensor, moisture sensor, and glucose sensor.

22. The method according to claim 1, additionally comprising the step
02.′ storing the recorded data in a memory integrated in the hearing aid.

23. The method according to claim 1, wherein the choice of arrangement is made via the interface, and wherein the choice of arrangement reaches the interface via wireless communication from an external computer device connected to the hearing aid.

24. The method according to claim 1, wherein the method can be automated according to the user's specifications.

25. The method according to claim 1, wherein the method is executed automatically when an activity state of the wearer changes, wherein the activity state of the wearer is continuously recorded via an acceleration sensor.

26. The method according to claim 1, additionally comprising the steps
03.′ determining, from the recorded data of two or more sensors S₁, S₂-Sᵢ, a data pattern D₁, D₂-Dᵢ of the wearer of the hearing aid, wherein each sensor S₁, S₂-Sᵢ is associated with a corresponding sensor index SI₁, SI₂-SIᵢ,
03.″ calculating, by means of calculation means, at least one health indicator of the wearer as a function of the at least one data pattern and a multidimensional pattern of health-related data, wherein a first dimension is time and at least a second dimension is the sensor index SI₁, SI₂-SIᵢ, wherein the health indicator and/or the data pattern and/or the recorded data are transmitted to the interface, and
03.′″ checking, by means of checking means, a plausibility of the health indicator using the sensor data from sensors S₁, S₂-Sᵢ with different modalities.

27. The method according to claim 26, wherein the multidimensional pattern of health-related data is a wearer-specific pattern, which is provided by the steps
1. recording sensor data of a wearer of a hearing aid in a first time interval, wherein the sensor data are data from one or more sensors S₁-Sᵢ, arranged in a hearing aid of a wearer, for recording health-related data,
2. determining, from the sensor data of the one or more sensors S₁-Sᵢ, a respective data pattern D₁-Dᵢ of the wearer in the first time interval,
3. recording sensor data of the wearer of a hearing aid in at least one further time interval,
4. determining, from the sensor data of the one or more sensors S₁-Sᵢ, a respective data pattern D₁-Dᵢ of the wearer in the at least one further time interval,
5. calculating, by means of calculation means, an average data pattern DD₁-DDᵢ of the user from the data patterns determined in step (b) and step (d) in order to obtain the multidimensional pattern of health-related data of the wearer.

28. The method according to claim 26, wherein the multidimensional pattern of health-related data is a reference table of general, physiological data.

29. The method according to claim 26, wherein the at least one health indicator specifies a measured value of the quality of the measured values and also of the health of the wearer in the time interval.

30. The method according to claim 26, wherein calculation means are arranged in the hearing aid, in a mobile computer unit and/or in a central processing unit.

31. The method according to claim 1, wherein the interface is arranged in a mobile computer unit and/or in a central processing unit.

32. The method according to claim 1, wherein the method comprises an authentication step and/or an encryption step.

33. A data processing system, comprising means for executing a method for providing data for an interface, wherein the interface is configured for computer-implemented health monitoring by means of at least one electronic apparatus integrated in a hearing aid, which method comprises:
01. providing an arrangement of one or more sensors S1-Si, which are arranged in a hearing aid and are designed to record health-related data, for selection by a user of the health-related data,
02. recording a user's choice of arrangement,
03. recording health-related data of a wearer of the hearing aid by means of the one or more sensors, selected by the user, in a time interval,
04. transmitting the recorded data to an interface.

34. A computer program, comprising instructions which, when the program is run on a computer, cause the computer to execute a method for providing data for an interface, wherein the interface is configured for computer-implemented health monitoring by means of at least one electronic apparatus integrated in a hearing aid, which method comprises:
01. providing an arrangement of one or more sensors S1-Si, which are arranged in a hearing aid and are designed to record health-related data, for selection by a user of the health-related data,
02. recording a user's choice of arrangement,
03. recording health-related data of a wearer of the hearing aid by means of the one or more sensors, selected by the user, in a time interval,
04. transmitting the recorded data to an interface.

35. A computer-readable storage medium, comprising instructions which, when the program is run on a computer, cause the computer to execute a method for providing data for an interface, wherein the interface is configured for computer-implemented health monitoring by means of at least one electronic apparatus integrated in a hearing aid, which method comprises:
01. providing an arrangement of one or more sensors S1-Si, which are arranged in a hearing aid and are designed to record health-related data, for selection by a user of the health-related data,
02. recording a user's choice of arrangement,
03. recording health-related data of a wearer of the hearing aid by means of the one or more sensors, selected by the user, in a time interval,
04. transmitting the recorded data to an interface.
